(19) United States
(12) Patent Application Publication
(10) Pub. No.: US 2016/0175239 A1
(43) Pub. Date: Jun. 23, 2016

(54) SMALL PARTICLE SIZE RENEWABLE TRIGLYCERIDE WAXES FOR USE IN CONSUMER AND INDUSTRIAL APPLICATIONS

(71) Applicant: H R D CORPORATION, Houston, TX (US)

(72) Inventors: Aziz Hassan, Houston, TX (US); Abbas Hassan, Houston, TX (US); Gregory G. Borsinger, Chatham, NJ (US)

(73) Assignee: H R D CORPORATION

(21) Appl. No.: 14/978,738

(22) Filed: Dec. 22, 2015

Related U.S. Application Data
(60) Provisional application No. 62/095,654, filed on Dec. 22, 2014.

(51) Int. Cl. A61K 8/92; A61K 8/04; C09D 11/00; A61Q 11/00; A61Q 1/10; C11D 3/20; A61K 8/02; A61Q 15/00

(52) U.S. Cl. CPC A61K 8/922; A61K 8/0241; A61K 8/04; A61Q 15/00; A61Q 11/00; A61Q 1/10; C11D 3/2093; C09D 11/00; A61K 2800/412; A61K 2800/591

(57) ABSTRACT

A composition comprises a plurality of microbeads dispersed within a base material. The plurality of microbeads is formed from a material comprising a renewable triglyceride wax having an I.V. of less than 70. The base material may be selected from the group consisting of soap, toothpaste, deodorant, mascara, ink, and cosmetics. The renewable triglyceride wax may be derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof.

### CROSS-REFERENCE TO RELATED APPLICATIONS

This application claims the benefit under 35 U.S.C. §119(e) of U.S. Provisional Patent Application 62/095,654 filed Dec. 22, 2014, the disclosure of which is hereby incorporated herein by reference.

### BACKGROUND

This disclosure relates generally to compositions including microbeads and micronized natural waxes derived from naturally sourced, renewable waxes that are synthesized from animal and vegetable matter. More specifically, this disclosure relates to compositions that include biodegradable microbeads as well as methods and apparatus for forming these compositions derived from triglycerides.

Microbeads, or other micronized particles, of non-degradable plastics such as polyethylene, polypropylene, and polystyrene have found wide use in consumer and industrial applications including personal care products, deodorants, cosmetics, skin care products, abrasive cleanser products, sand blasting, and other industrial applications. Microbeads are generally commercially available in particle sizes from 10 μm to 1 mm (1000 μm). As microbeads are often too small to be removed during conventional wastewater treatment, the use of non-degradable plastic microbeads has led to environmental concerns due to the persistence of the non-degradable material.

Waxes, both synthetic and naturally derived, can be used to form microbeads or in micronized or fine powder form. The particles in micronized waxes are further suspended in an organic or inorganic liquid such as water or oil. The process of micronizing wax particles is an energy intensive method that is often done in jet mills. To avoid melting of wax during milling, low temperature cryogenic gases are often employed to reduce processing temperatures thereby further increasing costs. Other techniques are also used to provide a fine-particle size wax in a liquid. Ball milling and pebble milling of waxes in a suitable solvent are often used to create a suspension of wax in solvent. These are also energy intensive techniques and require a suitable solvent (i.e. toluene, xylene) that may be undesirable for environmental and other reasons. Atomization is another technique for producing a small particle size spherical wax particle.

Micronized waxes are incorporated into scrubs to act as a mild abrasive for cleansers. European Patent EP0759971 describes the use of micronized wax in bar soap. Micronized waxes are also used in cosmetic foundation applications to provide a smooth fill for wrinkles, and for moisture resistance. For example, United States Patent Application No. 2004/0018250 describes some of the uses and benefits of waxes in cosmetic and pharmaceutical applications and highlights the undesirable effect of utilizing emulsifiers to incorporate wax in these applications. The inventor cites the use of high pressure to produce an emulsion. However, it is disclosed that the wax be incorporated into an organic carrier/solvent (e.g. silicone/glycol) prior to emulsifying. Thus, the final product is an emulsion and not a true dispersion of wax in a fluid. Micronized waxes are used in deodorants (U.S. Pat. No. 4,822,603) to aid in the pay-off and feel of a deodorant stick.

Micronized waxes are also used in ink, toner and coating and powder coating applications where they are dispersed in a vehicle to provide mar and abrasion resistance in such applications as over print varnish. Industrial and consumer coating applications also use micronized waxes to modify viscosity properties of paints as well as for their ability to reduce mar and abrasion. Pre dispersed micronized waxes are often made as a concentrate and then added to a coating. The micronized wax imparts a thixotropic viscosity profile to coatings that is beneficial in application of the coating as well as in reducing slump of the coating once applied. Microbeads also provide anti-blocking properties for such items as adhesives, rubber and coating applications.

Thus, there is a need for compositions including biodegradable microbeads as well as methods and apparatus for producing these compositions.

### BRIEF SUMMARY OF THE DISCLOSURE

A composition comprises a plurality of microbeads dispersed within a base material. The plurality of microbeads is formed from a material comprising a renewable triglyceride wax having an I.V. of less than 70. In certain embodiments, the base material is selected from the group consisting of soap, toothpaste, deodorant, mascara, ink, and cosmetics. In certain embodiments, the renewable triglyceride wax is derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof. In certain embodiments, the renewable triglyceride wax has an I.V. of less than 30. In certain embodiments, the renewable triglyceride wax has an I.V. of less than 10. In certain embodiments, the plurality of microbeads has an average diameter of less than 3 mm. In certain embodiments, the plurality of microbeads has an average diameter of less than 1 mm. In certain embodiments, the plurality of microbeads has an average diameter of less than 5 μm.

In other embodiments, a method comprises forming a plurality of microbeads from a material comprising a renewable triglyceride wax having an I.V. of less than 70, and dispersing the plurality of microbeads in a base material. In certain embodiments, the base material is selected from the group consisting of soap, toothpaste, deodorant, mascara, ink, and cosmetics. In certain embodiments, the renewable triglyceride wax is derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof. In certain embodiments, the renewable triglyceride wax has an I.V. of less than 30. In certain embodiments, the renewable triglyceride wax has an I.V. of less than 10. In certain embodiments, the plurality of microbeads has an average diameter of less than 3 mm. In certain embodiments, the plurality of microbeads has an average diameter of less than 1 mm

In other embodiments, a composition comprises a plurality of microbeads dispersed within the base material, wherein the plurality of microbeads comprise a renewable triglyceride wax having an I.V. of less than 70 and wherein the plurality of microbeads have an average diameter of less than 3 mm. In certain embodiments, the base material is selected from the group consisting of soap, toothpaste, deodorant, mascara, ink, and cosmetics. In certain embodiments, the renewable triglyceride wax is derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof. In certain embodiments, the renewable triglyceride wax has an I.V. of less than 10 and the plurality of microbeads have an average diameter of less than 5 μm.

### DETAILED DESCRIPTION

As used herein, the term “microbead” refers to micronized product, including microbeads, microspheres, and other particles that can be either spherical or irregularly shaped. Microbeads may have a diameter between about 1 μm to about 3 mm (3000 μm). Microbeads may be produced by either mechanical grinding or milling or by atomization that involves expanding a pressurized molten stream of triglyceride through a nozzle. Microbeads may also be produced by means of high shear devices.

As used herein, the term “renewable triglyceride wax” refers to a plant or animal-based triglyceride substance which has a solid, wax-like consistency at ambient conditions (72° F., 50% relative humidity). The term includes vegetable oils which have been partially or fully hydrogenated or fractionated to generate a solid, wax-like consistency, and other plant-based substances such as carnauba wax and candelilla wax which have a solid, wax-like consistency without requiring hydrogenation. Animal based triglycerides are also included. Animal based products are triglycerides usually produced in rendering operations.

As used herein, “iodine value” or “I.V.” is the number of grams of iodine that an unsaturated compound or blend will absorb in a given time under arbitrary conditions. A low iodine value implies a high level of saturation, and vice versa. The iodine value can be determined by the WIJS method of the American Oil Chemists' Society (A.O.C.S. Cd 1-25).

Certain embodiments of the present invention include a composition having a plurality of microbeads dispersed in within a base material. The microbeads are formed from a material comprising renewable triglyceride wax, which is used as an alternative to petroleum derived wax (both paraffin and synthetic ethylene derived waxes). The base material may be any of a number of substances used in a variety of applications including, but not limited to, chemical, cosmetic, food preparation, personal hygiene, pharmaceuticals and printing applications. Renewable triglyceride waxes can be obtained from a number of natural sources, including oils derived from soybean, soy stearine, stearine, castor, corn, cottonseed, rape, canola, sunflower, palm, palm kernel, coconut, crambe, linseed, peanut, and blends thereof.

Renewable triglyceride waxes include a broad class of organic esters and waxy compounds, which span a variety of chemical structures and display a broad range of melting temperatures. Often the same compound may be referred to as a wax, fat, or oil depending on the ambient temperature, the chain lengths of the esterified fatty acids, and their degree of saturation or unsaturation. By whatever name it is called, the choice of a renewable triglyceride wax for a particular application is often determined by whether it is a liquid or solid at the temperature of the product with which it is to be used.

Among the factors that determine whether a wax is a liquid or a solid at a given temperature are properties such as the degree of saturation or unsaturation of the components of the wax, primarily the fatty acids, and a property such as the iodine number, or iodine value (“I.V.”). The I.V. measures the amount of iodine absorbed in a given time by a compound or mixture, and the I.V. is thus a measure of the unsaturation, or number of double bonds, in that compound or mixture. Generally, the greater the degree of saturation and the longer the chain length of the esterified fatty acids, the higher the melting point. Similarly, the lower the I.V. of the compound, the harder, and more solid it will be at a particular temperature.

Renewable triglyceride waxes can be synthetically hydrogenated, using methods known to those skilled in the art, to have low or very low iodine values. Renewable triglyceride waxes having a low iodine value of between about 0 and 70, or between about 0 and 30, or between o and 10, may be produced by hydrogenation of a commercial oil, such as oils of soybean, soy stearine, stearine, castor, corn, cottonseed, rape, canola, sunflower, palm, palm kernel, coconut, crambe, linseed, peanut, and blends thereof. These oils may also be produced from genetically engineered plants to obtain low I.V. oil with a high percentage of fatty acids.

Embodiments of the present invention employ a highly hydrogenated triglyceride renewable triglyceride wax with an I.V. close to zero, thereby rendering the compound more thermally stable. Renewable triglyceride waxes can be chosen from those having an I.V. of between 0 to about 70, of between 0 to about 30, or of between 0 to about 10.

Microbeads may be produced by a number of processes including, atomizing, jet milling, hammer milling, dispersion, and other methods known in the art. Certain methods used for producing conventional non-vegetable wax microbeads may be equally effective in producing microbeads using materials comprising renewable triglyceride waxes. Microbeads formed using mechanical devices, such as hammer and jet milling, may use renewable triglyceride waxes having a high degree of hardness as well as high melting point in order to reduce the need for excessive cooling to avoid melting due to the heat generated in the milling process. Microbeads formed using atomizing or dispersion methods may use renewable triglyceride waxes having a low viscosity and sharp melting point to aid in atomization and quenching of the microbeads.

In general, certain compositions may include microbeads having a size from about 1 μm to about 3 mm, from 1 μm up to about 1 mm, or from about 1 μm up to about 5 μm. The desired particle size and shape of the particle usually is a factor of the end use application. In many coating applications, it may be desirable to use microbeads having a diameter from about 1 μm up to about 5 μm. Larger, non-spherical microbeads may be desirable in applications where the abrasive nature of the microbead is a factor, such as in cleaning products, bar soap, and other mildly abrasive products.

Microbeads formed from materials comprising renewable triglyceride wax may find use in many industrial and personal care applications when dispersed in base materials including, but not limited to, soaps, cleansers, toothpastes, cosmetics, and deodorant. In particular, microbeads can provide mild abrasiveness when dispersed in base materials such as bar soap, liquid hand soap, body lotions, toothpastes, and other cleaning materials. Microbeads can also provide controlled distribution (pay-off) when used in personal care items such as stick deodorants. In mascara and other cosmetics, microbeads can provide a filling media for wrinkles. Microbeads can also be used as thickeners in cosmetic and soap applications.

In certain applications, the renewable triglyceride wax may be combined with other materials to enhance performance and/or manufacturability. Materials including renewable triglyceride waxes may also include polyethylene (PE) waxes, c) polytetrafluoroethylene (PTFE) waxes, polypropylene (PP) waxes, amide waxes, Fischer-Tropsch (FT) paraffins, montan waxes, macrocrystalline and microcrystalline paraffins, polar polyolefin waxes, sorbitan esters, polyamides, polyolefins, PTFE, wetting agents, and silicates.

Microbeads can also be utilized in toner and ink applications as well as coatings. In particular, microbeads can be used in over-print varnish applications where the microbeads aid in reducing mar and abrasion as well as modify the slip properties of the coating. Microbeads can also provide anti-blocking properties for such items as adhesives, rubber and coating applications. Microbeads may be natural colored or compounded with dyes or pigments to provide coloration in any of the above applications. Microbeads can also be utilized as anti-sag, thickening and leveling agents in coating formulations. Microbeads have also been applied in down-hole oil drilling applications to control fluid loss and aid lubrication.

Microbeads formed from materials comprising renewable triglyceride wax may find use as an abrasive in applications including, industrial abrasives, such as those used for as scouring pastes or for polishing leather and woods, tumbling media, such as those used for polishing or finishing small parts. Microbeads can also be used in dental pastes to clean and polish tooth enamel, in cosmetic scrubs as an exfoliant, cleanser, or smoothing material, and in hand cleansers as a mild abrasive. Certain microbeads can also be used as a mold release agent to coat the inside of a mold to prevent sticking or as a protective coating for seeds to prevent moisture damage and provide lubrication during seed drilling operations.

While the disclosure is susceptible to various modifications and alternative forms, specific embodiments thereof are shown by way of example in the drawings and description. It should be understood, however, that the drawings and detailed description thereto are not intended to limit the disclosure to the particular form disclosed, but on the contrary, the intention is to cover all modifications, equivalents and alternatives falling within the spirit and scope of the present disclosure.

What is claimed is:

1. A composition comprising:
a base material; and
a plurality of microbeads dispersed within the base material, wherein the plurality of microbeads is formed from a material comprising a renewable triglyceride wax having an I.V. of less than 70.

2. The composition of claim 1, wherein the base material is selected from the group consisting of soap, toothpaste, deodorant, mascara, ink, and cosmetics.

3. The composition of claim 1, wherein the renewable triglyceride wax is derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof.

4. The composition of claim 1, wherein the renewable triglyceride wax has an I.V. of less than 30.

5. The composition of claim 1, wherein the renewable triglyceride wax has an I.V. of less than 10.

6. The composition of claim 1, wherein the plurality of microbeads has an average diameter of less than 3 mm.

7. The composition of claim 1, wherein the plurality of microbeads has an average diameter of less than 1 mm.

8. The composition of claim 1, wherein the plurality of microbeads has an average diameter of less than 5 μm.

9. A method comprising:
forming a plurality of microbeads from a material comprising a renewable triglyceride wax having an I.V. of less than 70; and
dispersing the plurality of microbeads in a base material.

10. The method of claim 9, wherein the base material is selected from the group consisting of soap, toothpaste, deodorant, mascara, ink, and cosmetics.

11. The method of claim 9, wherein the renewable triglyceride wax is derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof.

12. The method of claim 9, wherein the renewable triglyceride wax has an I.V. of less than 30.

13. The method of claim 9, wherein the renewable triglyceride wax has an I.V. of less than 10.

14. The method of claim 9, wherein the plurality of microbeads has an average diameter of less than 3 mm.

15. The method of claim 9, wherein the plurality of microbeads has an average diameter of less than 1 mm.

16. The method of claim 9, wherein the plurality of microbeads has an average diameter of less than 5 μm.

17. A composition comprising:
a base material; and
a plurality of microbeads dispersed within the base material, wherein the plurality of microbeads comprise a renewable triglyceride wax having an I.V. of less than 70 and wherein the plurality of microbeads have an average diameter of less than 3 mm.

18. The composition of claim 17, wherein the base material is selected from the group consisting of soap, toothpaste, deodorant, mascara, ink, and cosmetics.

19. The composition of claim 17, wherein the renewable triglyceride wax is derived from oils selected from the group consisting of soybean, soy stearine, stearine, castor, corn, cottonseed, rape, canola, sunflower, palm, palm kernel, coconut, crambe, linseed, peanut, or fats, such as animal fats, including lard and tallow, and blends thereof.

20. The composition of claim 17, wherein the renewable triglyceride wax has an I.V. of less than 10 and the plurality of microbeads has an average diameter of less than 5 μm.
